(19) United States
(12) Patent Application Publication
(10) Pub. No.: US 2024/0043905 A1
(43) Pub. Date: Feb. 8, 2024

(54) SAMPLE PRETREATMENT KIT AND METHOD FOR DETECTING VIRUS INFECTIVITY

(71) Applicant: Delta Electronics Int’l (Singapore) Pte Ltd, Singapore (SG)

(72) Inventors: Yong Zhang, Singapore (SG); Jia Hui Esther Boey, Singapore (SG); Quanke Long, Wuhan City (CN); Kun-Nan Tsai, Taipei (TW); Weishi Zhang, Singapore (SG); Jiaojiao Li, Wuhan City (CN); Xiaozhi Zhao, Wuhan City (CN)

(21) Appl. No.: 18/229,072

(22) Filed: Aug. 1, 2023

(30) Foreign Application Priority Data
Aug. 8, 2022 (CN) 202210943119.7

(51) Int. Cl. C12Q 1/6806; C12Q 1/70

(52) U.S. Cl. CPC C12Q 1/6806; C12Q 1/70

(57) ABSTRACT

A sample pretreatment kit for detecting virus infectivity includes a photoactivatable dye capable of intercalating into a nucleic acid, and a nuclease capable of degrading the nucleic acid. The photoactivatable dye includes PMA dye, PMAxx dye, EMA, platinum compounds, or palladium compounds. The method for detecting virus infectivity includes steps of: (a) dividing a clinical sample into a test sample and a control sample; (b) treating the test sample with a photoactivatable dye and a nuclease; (c) exposing the test sample to a light for photoactivation; (d) amplifying a target nucleic acid in the test sample and the control sample; and (e) determining virus infectivity based on amplification results of the test sample and the control sample.

### CROSS-REFERENCE TO RELATED APPLICATION

This application claims priority to China Patent Application No. 202210943119.7, filed on Aug. 8, 2022, the entire contents of which are incorporated herein by reference for all purposes.

### FIELD OF THE INVENTION

The present invention relates to sample pretreatment kit and method, and more particularly to sample pretreatment kit and method for detecting virus infectivity.

### BACKGROUND OF THE INVENTION

In recent years, the new incidence rate of the respiratory infectious disease ranks first among all diseases in global regions including China, Taiwan, Southeast Asia, Europe and the United States. This kind of disease is generally caused by various microorganisms which reach human bodies through respiratory organs such as nose, throat, and trachea, thereby causing related infectious diseases. The respiratory infectious disease spreads very quickly and widely, which causes a great impact on human health. Since it is difficult to control once the disease spreads, the respiratory infectious disease is a very serious and harmful infectious disease. The new coronavirus (COVID-19) pneumonia that originated at the end of 2019 is such a highly contagious disease with a high mortality rate. At present, almost no region in the world is spared. For example, India becomes one of the most serious regions. A large number of infected people have caused the shortage of medical resources, which is a huge disaster for the entire country. Therefore, the prevention and control of the respiratory infectious disease must be done well, and the early diagnosis of the disease is the top priority of the prevention work.

Clinically, the diagnosis of the respiratory infectious disease is not only based on clinical symptoms and signs, but also needs to be confirmed by laboratory tests. In tradition, the isolation and culture method is mainly used for virus detection, but this method is time-consuming and requires strict experimental conditions. For example, COVID-19 virus needs to be carried out in a laboratory above the BSL level (P3) level, and the operation is quite complicated.

Taking COVID-19 as an example, another clinical detection method is the antibody detection based on immunoassay and using serum as the sample. The antibody detection is to detect a serological immune response after the human is infected with the COVID-19 coronavirus. The immunoglobulin IgM antibodies are first produced in about 5 to 7 days, and then the IgG antibodies are produced in about 10 to 15 days. Namely, the IgM and IgG antibodies are produced after a certain incubation period since the antigens enter the human body, and during this period, the IgM and IgG antibodies cannot be detected in serum. Therefore, the antibody detection cannot be used as the gold standard for COVID-19 detection due to the window period. Nevertheless, the antibody detection was still widely used in the early stage of the epidemic owing to its low detection cost and simple operation. With the further outbreak of the epidemic, in order to facilitate large-scale screening in primary hospitals and home self-assessment, the COVID-19 antigen detection is adapted. However, the sensitivity and the specificity of the antigen detection are relatively low, and false negatives are prone to occur, so the antigen detection still cannot be used as the gold standard for diagnosis. The polymerase chain reaction (PCR) or the reverse transcription polymerase chain reaction (RT-PCR for RNA virus because RNA must be reverse transcribed into DNA before PCR) has extremely high sensitivity, which can greatly advance the window period for detection and thus enable the diagnosis in the early stage of disease development to facilitate subsequent treatment plans. Therefore, PCR/RT-PCR is recommended by various countries and the world health organization (WHO) as the gold standard for COVID-19 detection, and it is preferably qPCR/RT-qPCR for real-time quantitative detection.

The WHO once stated that the patients who were infected with COVID-19 and then declared to have recovered may still be tested positive later because their bodies are still eliminating dead lung cells, so they are not reinfected. During the recovering of the lung, some parts of dead cells will come out. These lung fragments cause the test to be still positive, but they are not contagious viruses, so it is not a relapse and this is actually part of the recovering process. In view of the PCR principle, the nucleic acid (RNA) of the virus, rather than the virus itself, is amplified and detected, so it is impossible to distinguish whether the virus is dead or alive, or whether the virus is infectious. Even in recovered patients, the RNA fragments of dead viruses are still possible to be positively tested, but these recovered patients themselves are not contagious. The positive test result obtained by this method will lead to further quarantine of the recovered patients. Except causing great psychological pressure to the patients, it will also increase the social burden. Therefore, a PCR detection method that can distinguish whether the virus in the clinical sample is infectious or not is of great significance.

### SUMMARY OF THE INVENTION

An object of the embodiments of the present disclosure is to provide sample pretreatment kit and method for detecting virus infectivity, so as to distinguish whether the virus in the clinical sample is infectious or not, and further provide clinical diagnostic information for virus-infected patient's management and therapy.

In accordance with an aspect of the embodiments of the present disclosure, a sample pretreatment kit for detecting virus infectivity is provided. The sample pretreatment kit includes a photoactivatable dye capable of intercalating into a nucleic acid, and a nuclease capable of degrading the nucleic acid. The photoactivatable dye includes PMA dye, PMAxx dye, EMA, platinum compounds, or palladium compounds.

In an embodiment, a final reaction concentration of the PMA dye or the PMAxx dye is ranged 100 to 2000 μM.

In an embodiment, the nuclease includes a ribonuclease, and a final reaction concentration of the ribonuclease is ranged 0.1 to 100 ng/μl.

In accordance with another aspect of the embodiments of the present disclosure, a method for detecting virus infectivity is provided. The method includes steps of: (a) dividing a clinical sample into a test sample and a control sample; (b) treating the test sample with a photoactivatable dye and a nuclease, wherein the photoactivatable dye includes PMA dye, PMAxx dye, EMA, platinum compounds, or palladium compounds; (c) exposing the test sample to a light for photoactivation; (d) amplifying a target nucleic acid in the test sample and the control sample; and (e) determining virus infectivity based on amplification results of the test sample and the control sample.

In an embodiment, a final reaction concentration of the PMA dye or the PMAxx dye is ranged 100 to 2000 μM.

In an embodiment, the nuclease includes a ribonuclease, and a final reaction concentration of the ribonuclease is ranged 0.1 to 100 ng/μl.

In an embodiment, a time for the photoactivation is ranged 2 to 30 minutes.

In an embodiment, the target nucleic acid is RNA, and the target nucleic acid is reverse-transcribed into DNA by using at least one pair of primers and then amplified.

In an embodiment, before performing nucleic acid amplification in the step (d), the test sample and the control sample are subject to nucleic acid extraction.

In an embodiment, a criterion for determining virus infectivity in the step (e) is that when a Cq value of the test sample is larger than that of the control sample and there is a significant difference, or when there is no typical amplification curve and no Cq value for the test sample, it is determined that there is no infectious virus in the clinical sample.

The above contents of the present invention will become more readily apparent to those ordinarily skilled in the art after reviewing the following detailed description and accompanying drawings, in which:

### BRIEF DESCRIPTION OF THE DRAWINGS

FIG. 1A and FIG. 1B show the qPCR amplification curves of the OFR1ab gene of COVID-19 after PMAxx and RNase treatment;

FIG. 2A and FIG. 2B show the qPCR amplification curves of the E gene of COVID-19 after PMAxx and RNase treatment;

FIG. 3 shows the qPCR amplification curves of the OC43 live virus and the OC43 dead virus after RNase treatment; and

FIG. 4A and FIG. 4B show the qPCR amplification curves of the OC43 live virus and the OC43 dead virus after treatment of the sample pretreatment kit.

### DETAILED DESCRIPTION OF THE PREFERRED EMBODIMENT

The present invention will now be described more specifically with reference to the following embodiments. It is to be noted that the following descriptions of the embodiments of this invention are presented herein for purpose of illustration and description only; it is not intended to be exhaustive or to be limited to the precise form disclosed.

The embodiments of the present disclosure provide sample pretreatment kit and method for detecting virus infectivity, so as to distinguish whether the virus in the clinical sample is infectious or not, and further provide clinical diagnostic information for virus-infected patient's management and therapy. The method uses a photoactivatable or photosensitive dye, which is a nucleic acid intercalating dye, that selectively enters cells with compromised or incomplete cell membranes, whereas an intact cell membrane presents a barrier for this molecule. Once inside a (dead) cell, the dye intercalates into the cell's nucleic acid to which it is believed to covalently crosslink after exposure to strong visible light due to the presence of an azide group, and this modification strongly inhibits nucleic acid amplification. At the same time when the cross-linking with the nucleic acid occurs, any unbound excess dye reacts with the water molecule, and the resulting hydroxylamine is no longer reactive, preventing reaction of the dye with nucleic acids extracted from intact cells. Therefore, by this mechanism, the clinical sample can be added with the photoactivatable dye capable of intercalating into the nucleic acid for pretreatment, and after photoactivation, the dye can preferably intercalate and covalently crosslink to the nucleic acids of the dead cells and thus prevent subsequent nucleic acid amplification from dead cells by PCR (including PCR, RT-PCR, qPCR, RT-qPCR).

In order to ensure that the virus infectivity can be determined according to the PCR result of the pretreated clinical sample, and further improve the effect of single dye treatment, the present disclosure further introduces other pretreatment method. Particularly, based on the differences of cell membrane integrity and nucleic acid exposure between dead and live viruses, the present disclosure also uses the nuclease to degrade the nucleic acids exposed by dead cells due to incomplete membranes, so as to further enhance the difference compared to the PCR result of the clinical sample without any treatment. In other words, the present disclosure combines the photoactivatable dye with light and the nuclease for degrading nucleic acids to detect the infectivity of the clinical sample. While ensuring the detection effect, the pretreatment methods can also be flexibly combined according to actual clinical application scenarios, so as to reduce the rigid demand for detection resource allocation and improve the operational convenience.

Specifically, the present disclosure provides the sample pretreatment kit and method for detecting virus infectivity in the clinical sample. After the clinical sample is pretreated with the sample pretreatment kit under certain conditions, the nucleic acids in the sample are extracted and detected by PCR. By comparing the difference of Cq value with the sample without any treatment, whether the virus in the clinical sample is infectious can be determined. The sample pretreatment kit includes the photoactivatable dye capable of intercalating into the nucleic acid and the nuclease capable of degrading the nucleic acid. After mixing the clinical sample with the sample pretreatment kit, the mixture is put into an illumination platform which provides light with a specific wavelength. By adjusting the parameters of the platform (such as LED brightness and illumination time), the test sample is photoactivated and the sample pretreatment is completed. The sample without pretreatment is used as a control sample. The test sample and the control sample are respectively subject to nucleic acid extraction and PCR detection, and the Cq value difference and the amplification curves of the test sample and the control sample are compared to determine whether there is infectious virus in the test sample.

If the sample contains dead viruses (i.e., non-infectious virus), the nuclease will degrade the nucleic acids exposed by the dead cells due to incomplete membranes, and the photoactivatable dye will enter the dead cells to modify the nucleic acids and inhibit nucleic acid amplification, which results that the PCR amplification will be delayed or no amplification will occur. Therefore, the criterion for determining virus infectivity based on the PCR amplification results is as the following. When the Cq value of the sample treated with the photoactivatable dye and the nuclease is larger than that of the untreated sample (ΔCq>0) and there is a significant difference, or when the PCR reaction of the sample treated with the photoactivatable dye and the nuclease has no typical amplification curve and no Cq value (the Cq value is greater than the critical value for detection), it indicates that there is no infectious virus in the sample.

Accordingly, the present disclosure provides the method for detecting virus infectivity, which includes the steps of: (a) dividing the clinical sample into the test sample and the control sample; (b) treating the test sample with the photoactivatable dye and the nuclease; (c) exposing the test sample to the visible light for photoactivation; (d) amplifying target nucleic acids in the test sample and the control sample; and (e) determining virus infectivity based on the amplification results of the test sample and the control sample.

In an embodiment, the photoactivatable dye includes propidium monoazide (PMA) dye or PMAxx dye, or dyes with similar functions, such as ethidium monoazide (EMA), platinum compounds, or palladium compounds. In some other embodiments, in order to enhance the binding of the photoactivatable dye to the dead virus, a reagent enhancer can be added to work together with the photoactivatable dye for reducing the cell membrane integrity of the dead virus, thereby facilitates the binding of the photoactivatable dye to the nucleic acid of the dead virus. The reagent enhancer may include a surfactant, such as sodium dodecyl sulfate (SDS), sodium lauroyl sarcosinate, dimethyl sulfoxide (DMSO), ethylenediaminetetraacetic acid (EDTA), Triton X-100, or Igepal CA-630.

In an embodiment, the nuclease includes a ribonuclease (RNase). For RNA viruses, such as COVID-19, the RNase pretreatment can degrade the nucleic acids exposed by the dead cells due to incomplete membranes. In another embodiment, for DNA viruses, the nuclease may include deoxyribonuclease (DNase), which is also used to degrade the nucleic acids exposed by the dead cells due to incomplete membranes.

In an embodiment, the nucleic acid amplification method is PCR, and preferably qPCR for real-time quantitative detection. When the detection target is an RNA virus, the RNA thereof is first reverse-transcribed into DNA before PCR, so the said PCR is essentially RT-PCR, and preferably RT-qPCR for real-time quantitative detection. In other words, the target nucleic acid is RNA, and the target nucleic acid is reverse-transcribed into DNA by using at least one pair of primers, and then amplified by qPCR.

In an embodiment, the clinical sample is divided into two parts, wherein one is the test sample, and the other is the control sample. The test sample is pretreated with the photoactivatable dye and the nuclease, and the control sample is added with sterilized water or buffer. In the embodiment, the control sample may be only added with sterilized water or buffer. The steps of pretreatment include the following. First, a certain volume of the liquid test sample is added into a microcentrifuge tube (e.g., Eppendorf tube) which is free of DNase and RNase. Subsequently, a certain volume and concentration of the RNase solution is added into the above tube. Also, a certain volume and concentration of the PMA or PMAxx solution is added into the above tube in a dark environment and all the liquids in the above tube are mixed well. Then, the above tube is placed into the illumination platform, and the brightness and the illumination time of the visible light LED on the illumination platform are adjusted, so as to expose the sample in the tube to the visible light for photoactivation for a certain period of time. For example, the wavelength of the visible light is ranged 450 to 480 nm, and the intensity of the visible light is ranged 85 to 250 mW, but not limited thereto.

In an embodiment, the final reaction concentration of the RNase is ranged 0.1 to 100 ng/μl, and preferably 1 to 10 ng/μl.

In an embodiment, the final reaction concentration of the PMA or PMAxx is ranged 100 to 2000 μM, and preferably 100 to 300 μM.

In an embodiment, the pretreatment time of the PMA or PMAxx is ranged 5 to 30 minutes.

In an embodiment, the photoactivation time is ranged 2 to 30 minutes, and preferably 5 to 10 minutes.

In an embodiment, before performing nucleic acid amplification, the sample may be subject to nucleic acid extraction, for example, using a spin column extraction method. Then, the extracted nucleic acids are used as the template for PCR detection.

The sample pretreatment kit and method for detecting virus infectivity in the clinical sample of the present disclosure will be further illustrated below with examples.

Example 1 used the RNA standard substances for the E gene and the OFR1ab gene of COVID-19 to examine the effect of the sample pretreatment kit. The sample in this example was operated as follows.

1. The RNA standard substance solutions in a concentration of 5×10⁴ copies/μl for both the E gene and the OFR1ab gene of COVID-19 were prepared, and the tests for the two genes were operated separately in the following steps.

2. 5 μl of the prepared standard substance solution was added into each microcentrifuge tube respectively, wherein one group was also added with a certain volume of the RNase solution (0.1 ng/μl), another group was added with a certain volume of the PMAxx solution (2 mM), and a further group was added with a certain volume of the mixed solution (RNase solution+PMAxx solution).

3. The microcentrifuge tubes were placed into the illumination platform for photoactivation for 10 minutes. At the same time, the sample without any reagent treatment was also subject to light as a control.

4. The microcentrifuge tubes were removed from the illumination platform, and the nucleic acids in the tubes were extracted. Then, 1 μl of the extracted nucleic acids were added into the qPCR reaction system and the Cq values were determined.

The following Table 1 shows the settings of each group in Example 1 (numbers are sampling volumes (μl)).

TABLE 1

| | RNA | | | | |
|---|---|---|---|---|---|
| | (5 × 10\hat{ }4 | PMAxx | RNase | | Total |
| Group | copies/μl) | (2 mM) | (0.1 ng/μl) | water | volume |
| PMAxx | 5 | 1 | — | 94 | 100 |
| RNase (0.1) | 5 | — | 1 | 94 | 100 |
| RNase (0.5) | 5 | — | 5 | 90 | 100 |
| PMAxx + RNase (0.1) | 5 | 1 | 1 | 93 | 100 |
| PMAxx + RNase (0.5) | 5 | 1 | 5 | 89 | 100 |
| Control | 5 | — | — | 95 | 100 |

*RNase (0.1) represents that the final concentration of RNase is 1 pg/μl, and the absolute dosage is 0.1 ng. RNase (0.5) represents that the final concentration of RNase is 5 pg/μl, and the absolute dosage is 0.5 ng. The sample that RNase acts on in this example is the RNA standard substance, not a real clinical sample, so RNase has a good effect at a very low concentration.

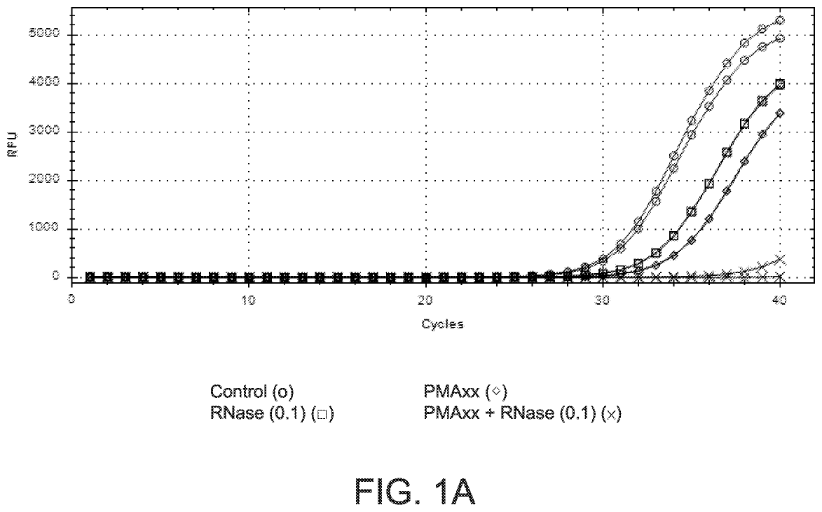

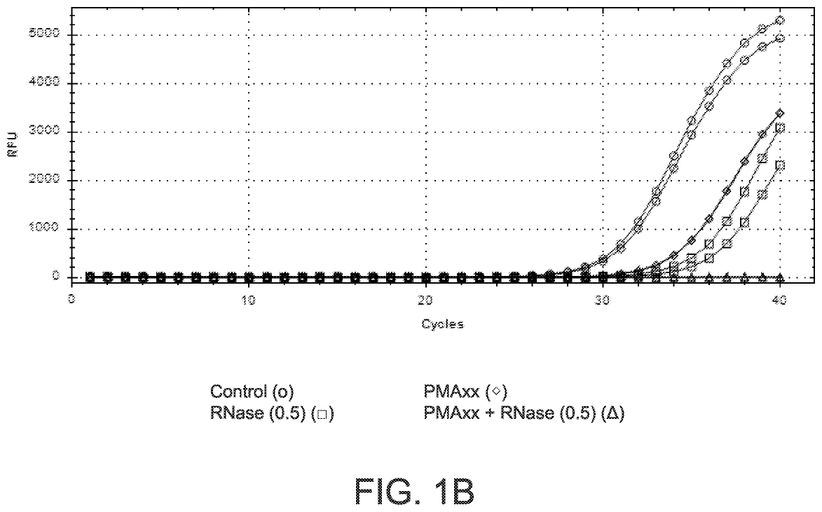

The following Table 2 shows the Cq values for detecting the OFR1ab gene of COVID-19 after PMAxx and RNase treatment. FIG. 1A shows the qPCR amplification curves of the OFR1ab gene of COVID-19 after PMAxx and RNase (0.1) treatment, and FIG. 1B shows the qPCR amplification curves of the OFR1ab gene of COVID-19 after PMAxx and RNase (0.5) treatment. From Table 2, FIG. 1A and FIG. 1B, it is clear that compared with the Cq value 29.70 of the control group, the groups of PMAxx treatment, RNase treatment, and PMAxx+RNase treatment had amplification delays (ΔCq>0) or no amplification occurred. The amplification inhibition efficiency for the group of PMAxx treatment was 89.82%, the amplification inhibition efficiency for the group of RNase (0.1) treatment was 77.38%, and the amplification inhibition efficiency for the group of PMAxx+RNase (0.1) treatment was greater than 98.73%. The results showed that the pretreatment with the photoactivatable dye and the nuclease collaboratively can indeed further enhance the effect of amplification inhibition, and increase the difference from the control group for further judgment. When the RNase concentration was increased, the amplification inhibition efficiency for the group of RNase (0.5) treatment was promoted to 97.97%, while the group of PMAxx+RNase (0.5) treatment had no amplification occurred (no typical amplification curve and no Cq value), which means the amplification inhibition efficiency reached 100%.

TABLE 2

| | | | | Inhibition |
|---|---|---|---|---|
| Group | Cq | Cq mean | ΔCq | efficiency |
| PMAxx | 32.98 | 32.98 | 3.28 | 89.82% |
| | 32.97 | | | |
| RNase (0.1) | 31.8 | 31.83 | 2.13 | 77.38% |
| | 31.86 | | | |
| PMAxx + RNase (0.1) | NA | — | >6.26 | >98.73% |
| | 35.96 | | | |
| RNase (0.5) | 35.69 | 35.29 | 5.59 | 97.97% |
| | 34.88 | | | |
| PMAxx + RNase (0.5) | NA | — | — | 100.00% |
| | NA | | | |
| Control | 29.65 | 29.70 | — | — |
| | 29.75 | | | |

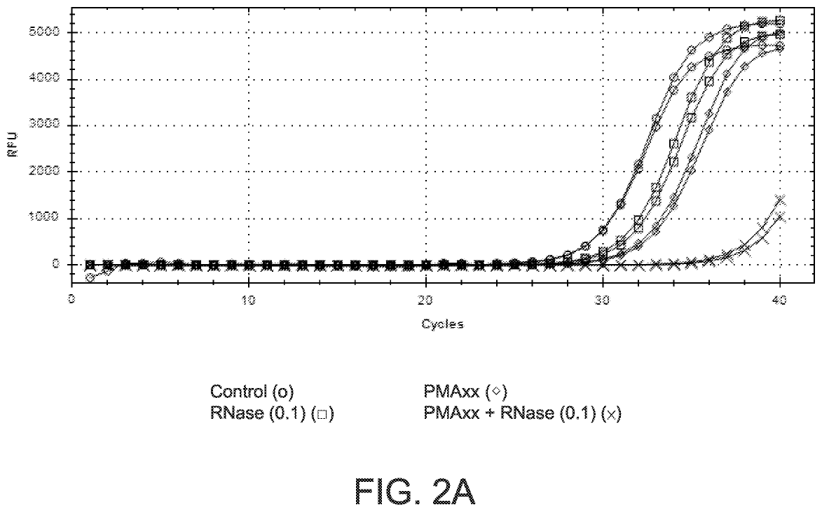

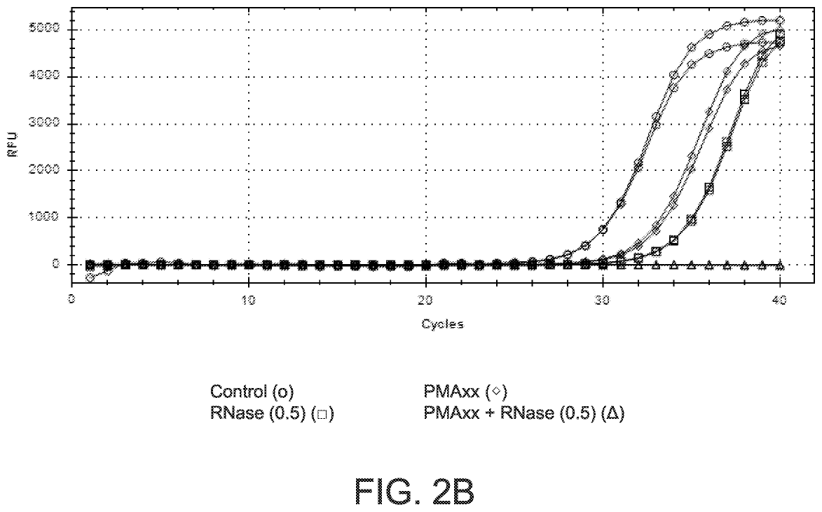

The following Table 3 shows the Cq values for detecting the E gene of COVID-19 after PMAxx and RNase treatment. FIG. 2A shows the qPCR amplification curves of the E gene of COVID-19 after PMAxx and RNase (0.1) treatment, and FIG. 2B shows the qPCR amplification curves of the E gene of COVID-19 after PMAxx and RNase (0.5) treatment. From Table 3, FIG. 2A and FIG. 2B, it is clear that compared with the Cq value 28.42 of the control group, the groups of PMAxx treatment, RNase treatment, and PMAxx+RNase treatment had amplification delays (ΔCq>0) or no amplification occurred. The amplification inhibition efficiency for the group of PMAxx treatment was 88.05%, the amplification inhibition efficiency for the group of RNase (0.1) treatment was 72.30%, and the amplification inhibition efficiency for the group of PMAxx+RNase (0.1) treatment was 99.87%. The results showed that the pretreatment with the photoactivatable dye and the nuclease collaboratively can indeed further enhance the effect of amplification inhibition, and increase the difference from the control group for further judgment. When the RNase concentration was increased, the amplification inhibition efficiency for the group of RNase (0.5) treatment was promoted to 96.18%, while the group of PMAxx+RNase (0.5) treatment had no amplification occurred (no typical amplification curve and no Cq value), which means the amplification inhibition efficiency reached 100%.

TABLE 3

| | | | | Inhibition |
|---|---|---|---|---|
| Group | Cq | Cq mean | ΔCq | efficiency |
| PMAxx | 31.53 | 31.47 | 3.05 | 88.05% |
| | 31.4 | | | |
| RNase (0.1) | 30.07 | 30.26 | 3.05 | 72.30% |
| | 30.45 | | | |
| PMAxx + RNase (0.1) | 37.82 | 37.91 | 3.05 | 99.87% |
| | 37.99 | | | |
| RNase (0.5) | 33.11 | 33.1 | 3.05 | 96.18% |
| | 33.09 | | | |
| PMAxx + RNase (0.5) | NA | — | — | 100.00% |
| | NA | | | |
| Control | 28.32 | 28.42 | — | — |
| | 28.51 | | | |

Example 2 used the human coronavirus HCoV-OC43 (hereinafter referred to as OC43 virus) to examine the effect of the RNase in the sample pretreatment kit. The sample in this example was operated as follows.

1. The OC43 virus culture was diluted 10 times with a viral transport medium (VTM) for further tests.

2. A certain volume of the virus dilution was incubated at 56° C. for 30 minutes to inactivate the virus.

3. A certain volume and concentration of the RNase solution was mixed with live virus and inactivated (dead) virus samples, respectively, and incubated at 32° C. in a metal bath at a speed of 200 rpm for 30 min. At the same time, the samples without RNase treatment were used as controls.

4. The nucleic acids were extracted. Then, 1 μl of the extracted nucleic acids were added into the qPCR reaction system and the Cq values were determined.

The following Table 4 shows the settings of each group in Example 2 (numbers are sampling volumes (μl)).

TABLE 4

| | OC43 | OC43 | RNase | | |
|---|---|---|---|---|---|
| | live | dead | (1000 | | Total |
| Group | virus | virus | ng/μl) | VTM | volume |
| Live virus control | 10 | — | — | 90 | 100 |
| Live virus + RNase (10) | 10 | — | 1 | 89 | 100 |
| Live virus + RNase (50) | 10 | — | 5 | 85 | 100 |
| Dead virus control | — | 10 | — | 90 | 100 |
| Dead virus + RNase (10) | — | 10 | 1 | 89 | 100 |
| Dead virus + RNase (50) | — | 10 | 5 | 85 | 100 |

*RNase (10) represents that the final concentration of RNase is 10 ng/μl, and RNase (50) represents that the final concentration of RNase is 50 ng/μl.

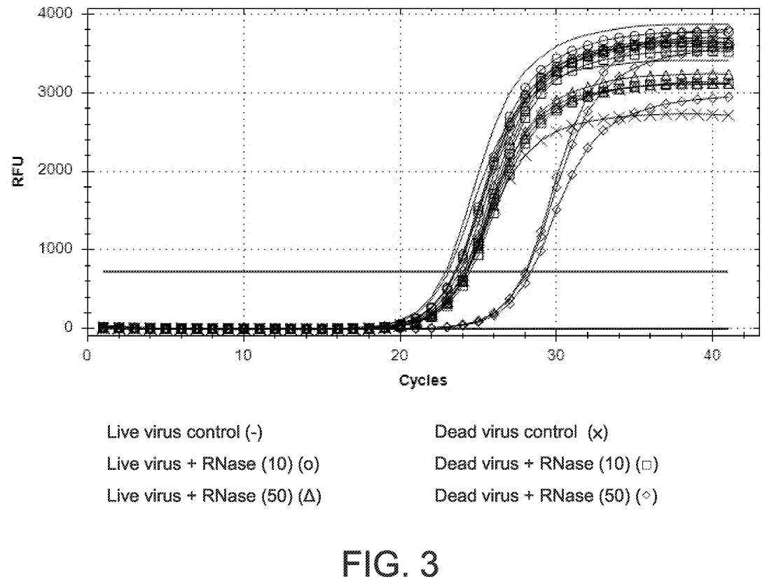

The following Table 5 shows the Cq values of the OC43 live virus and the OC43 dead virus after RNase treatment, and FIG. 3 shows the qPCR amplification curves of the OC43 live virus and the OC43 dead virus after RNase treatment. This example inactivated the live virus by heating (e.g., heating to 55-75° C. for 30 minutes), and most cells would die and were thus not infectious. From Table 5 and FIG. 3, it can be seen that in each group, only the group for dead virus treated with higher concentration (50 ng/μl) of RNase had a significant amplification delay. In other words, the pretreatment with RNase alone is not easy to directly distinguish whether there is non-infectious virus (dead virus) in the sample.

TABLE 5

| | | | | Inhibition |
|---|---|---|---|---|
| Group | Cq | Cq mean | ΔCq | efficiency |
| Live virus control | 23.04 | 22.94 | — | — |
| | 22.85 | | | |
| Live virus + RNase (10) | 23.47 | 23.47 | 0.53 | 30.9% |
| | 23.42 | | | |
| | 23.51 | | | |
| Live virus + RNase (50) | 24.19 | 24.16 | 1.22 | 57.3% |
| | 24.03 | | | |
| | 24.25 | | | |
| Dead virus control | 24.2 | 23.99 | — | — |
| | 24.05 | | | |
| | 23.73 | | | |
| Dead virus + RNase (10) | 24.4 | 24.26 | 0.27 | 17.2% |
| | 24.18 | | | |
| | 24.22 | | | |
| Dead virus + RNase (50) | 28.3 | 28.1 | 4.11 | 94.3% |
| | 28.03 | | | |
| | 27.96 | | | |

Example 3 used the OC43 virus to examine the effect of the sample pretreatment kit (PMAxx+RNase). The sample in this example was operated as follows.

1. The OC43 virus culture was diluted 10 times with the viral transport medium (VTM) for further tests.

2. A certain volume of the virus dilution was incubated at 56° C. for 30 minutes to inactivate the virus.

3. The above virus dilution was added into each microcentrifuge tube respectively, wherein one group was also added with a certain volume of the RNase solution (final concentration is 10 ng/μl), another group was added with a certain volume of the PMAxx solution (final concentration is 100 μM), and a further group was added with a certain volume of the mixed solution (RNase solution+PMAxx solution).

4. The microcentrifuge tubes were placed into the illumination platform for photoactivation for 10 minutes.

5. The microcentrifuge tubes were removed from the illumination platform, and the nucleic acids in the tubes were extracted. Then, 1 μl of the extracted nucleic acids were added into the qPCR reaction system and the Cq values were determined.

The following Table 6 shows the settings of each group in Example 3.

TABLE 6

| Sample | Group | PMAxx | Photoactivation | RNase |
|---|---|---|---|---|
| Dead | a: Dead virus control | − | − | − |
| virus | b: Dead virus + PMAxx | + | − | − |
| | c: Dead virus + PMAxx + | + | + | − |
| | Photoactivation | | | |
| | d: Dead virus control | − | − | − |
| | e: Dead virus + RNase + | + | − | + |
| | PMAxx | | | |
| | f: Dead virus + RNase + | + | + | + |
| | PMAxx + Photoactivation | | | |
| Live | A: Live virus control | − | − | − |
| virus | B: Live virus + PMAxx | + | − | − |
| | C: Live virus + PMAxx + | + | + | − |
| | Photoactivation | | | |
| | D: Live virus control | − | − | − |
| | E: Live virus + RNase + | + | − | + |
| | PMAxx | | | |
| | F: Live virus + RNase + | + | + | + |
| | PMAxx + Photoactivation | | | |

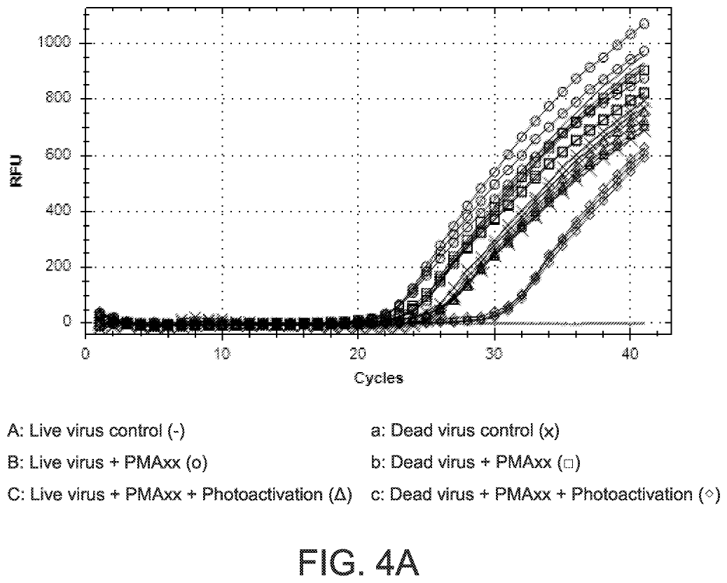

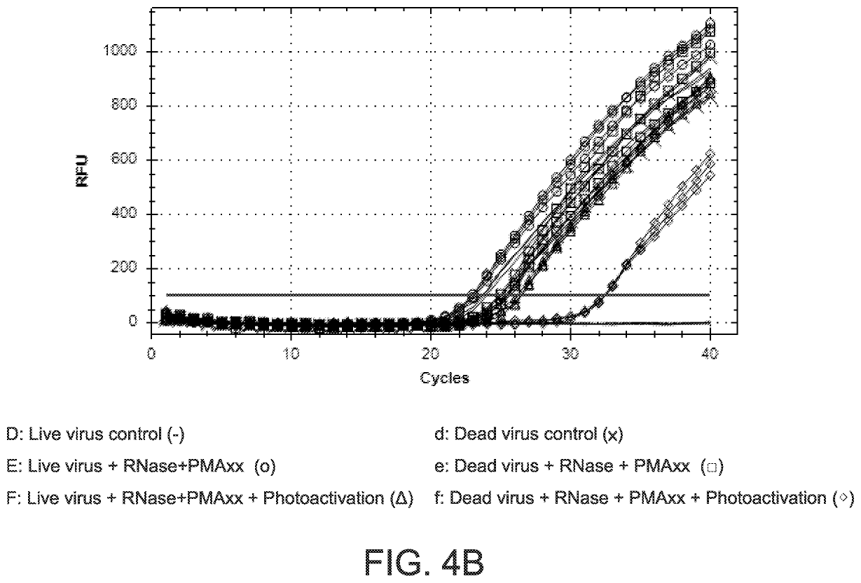

The following Table 7 shows the Cq values of the OC43 live virus and the OC43 dead virus after treatment of the sample pretreatment kit (PMAxx+RNase), and FIG. 4A and FIG. 4B show the qPCR amplification curves of the OC43 live virus and the OC43 dead virus after treatment of the sample pretreatment kit. It can be seen from Table 7 and FIG. 4A that, in the groups a˜c and A-C which were treated with PMAxx but without RNase, there was a significant amplification delay after the dead virus was treated with PMAxx and photoactivation, and the amplification inhibition efficiency was 97.40%. Further, it can be seen from Table 7 and FIG. 4B that, in the groups d-f and D-F which were treated with both RNase and PMAxx at the same time, there was a more significant amplification delay after the dead virus was treated with RNase, PMAxx and photoactivation, and the amplification inhibition efficiency was 99.70%, which can determine that there was no infectious virus in the sample. Comparing the amplification curves in FIG. 4A and FIG. 4B, it can be more clearly seen that the addition of RNase indeed further enhances the effect of amplification inhibition by PMAxx, and increase the difference from the control group, which contributes to judge whether there is infectious virus in the sample. In addition, the live virus sample may also contain some naturally dead cells. After being treated with RNase, PMAxx and photoactivation, the Cq value may be slightly increased but there is no significant difference, so it can be judged that the sample still contain infectious virus.

TABLE 7

| | | RT-qPCR | | |
|---|---|---|---|---|
| | | | | Inhibition |
| Sample | Group | Cq | ΔCq | efficiency |
| Dead virus | a | 24.26 | — | — |
| | b | 23.06 | −1.2 | — |
| | C | 29.52 | 5.26 | 97.40% |
| | d | 22.99 | — | — |
| | e | 23.37 | 0.38 | 23.30% |
| | f | 31.13 | 8.14 | 99.70% |
| Live virus | A | 23.19 | — | — |
| | B | 21.68 | −1.51 | — |
| | C | 24.94 | 1.75 | 70.50% |
| | D | 21.82 | — | — |
| | E | 21.18 | −0.64 | — |
| | F | 24.02 | 2.2 | 78.50% |

In conclusion, the present disclosure provides the sample pretreatment kit and method for detecting virus infectivity in the clinical sample, wherein the sample pretreatment kit includes the photoactivatable dye capable of intercalating into the nucleic acid and the nuclease capable of degrading the nucleic acid. When the sample contains dead virus (i.e., non-infectious virus), the nuclease degrades the nucleic acids exposed by the dead cells due to incomplete membranes, and the photoactivatable dye enters into the dead cells to modify the nucleic acids and inhibit the nucleic acid amplification, which results in that the PCR amplification is delayed or no amplification occurs. Therefore, when the clinical sample is pretreated with the sample pretreatment kit and subject to photoactivation, it can be determined whether the sample contains infectious virus by comparing the Cq value difference and the amplification curves of the pretreated and unpretreated samples according to the PCR amplification results. Particularly, the present disclosure further adds the nuclease to work with the photoactivatable dye together, so as to enhance the effect of amplification inhibition and increase the difference in PCR detection results from the unpretreated samples, which contributes to judge whether there is infectious virus in the sample. The PCR method combined with the sample pretreatment kit provided in the present disclosure has advantages of short detection time, strong specificity, reliable results, and simple result judgment. Therefore, the sample pretreatment kit and method of the present disclosure is applicable to re-examination on patients with infectious diseases after recovery and discharge, detection of infectious viruses in the environment, food safety detection, drinking water quality monitoring, live bacteria detection that requires time-consuming or difficult cultivation, etc., and it is of great significance to judge and formulate measures for virus elimination, prevention and control.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A sample pretreatment kit for detecting virus infectivity, comprising:
a photoactivatable dye capable of intercalating into a nucleic acid; and
a nuclease capable of degrading the nucleic acid,
wherein the photoactivatable dye comprises PMA dye, PMAxx dye, EMA, platinum compounds, or palladium compounds.

2. The sample pretreatment kit according to claim 1, wherein a final reaction concentration of the PMA dye or the PMAxx dye is ranged 100 to 2000 μM.

3. The sample pretreatment kit according to claim 1, wherein the nuclease comprises a ribonuclease.

4. The sample pretreatment kit according to claim 3, wherein a final reaction concentration of the ribonuclease is ranged 0.1 to 100 ng/μl.

5. A method for detecting virus infectivity, comprising steps of:
(a) dividing a clinical sample into a test sample and a control sample;
(b) treating the test sample with a photoactivatable dye and a nuclease, wherein the photoactivatable dye comprises PMA dye, PMAxx dye, EMA, platinum compounds, or palladium compounds;
(c) exposing the test sample to a light for photoactivation;
(d) amplifying a target nucleic acid in the test sample and the control sample; and
(e) determining virus infectivity based on amplification results of the test sample and the control sample.

6. The method according to claim 5, wherein a final reaction concentration of the PMA dye or the PMAxx dye is ranged 100 to 2000 μM.

7. The method according to claim 5, wherein the nuclease comprises a ribonuclease.

8. The method according to claim 7, wherein a final reaction concentration of the ribonuclease is ranged 0.1 to 100 ng/μl.

9. The method according to claim 5, wherein a time for the photoactivation is ranged 2 to 30 minutes.

10. The method according to claim 5, wherein the target nucleic acid is RNA, and the target nucleic acid is reverse-transcribed into DNA by using at least one pair of primers and then amplified.

11. The method according to claim 5, wherein before performing nucleic acid amplification in the step (d), the test sample and the control sample are subject to nucleic acid extraction.

12. The method according to claim 5, wherein a criterion for determining virus infectivity in the step (e) is that when a Cq value of the test sample is larger than that of the control sample and there is a significant difference, or when there is no typical amplification curve and no Cq value for the test sample, it is determined that there is no infectious virus in the clinical sample.
